(19) United States
(12) Patent Application Publication
(10) Pub. No.: US 2024/0181251 A1
(43) Pub. Date: Jun. 6, 2024

(54) METHOD OF CREATING AN ANTIOXIDANT, ANTIAPOPTOTIC, ANTIAGING, ANTI-INFLAMMATORY, ANTIBACTERIAL, ANTITOXIC, ENERGY METABOLISM REGULATING EFFECT AND BALANCING BLOOD PH BY CREATING CHEMICAL AND BIOLOGICAL REACTIONS IN HUMAN AND ANIMAL BODY

(71) Applicants: Sevil TUNC, Istanbul (TR); AVB BIYOTEKNOLOJI ANONIM SIRKETTI, Istanbul (TR); AVB BIYOTEKNOLOJI ANONIM SIRKETI, Istanbul (TR); BEZMIALEM VAKIF UNIVERSITESI, Istanbul (TR)

(72) Inventors: Serkan TUNC, Istanbul (TR); Sevil TUNC, Istanbul (TR); Rabia ATMACA, Istanbul (TR); Burak Tutkucan ODUNCU, Istanbul (TR); Ismail MERAL, Istanbul (TR); Savas USTUNOVA, Istanbul (TR)

(73) Assignees: Serkan TUNC, Istanbul (TR); Sevil TUNC, Istanbul (TR); AVB BIYOTEKNOLOJI ANONIM SIRKETI, Istanbul (TR); BEZMIALEM VAKIF UNIVERSITESI, Istanbul (TR)

(21) Appl. No.: 18/285,273

(22) PCT Filed: Feb. 18, 2022
(86) PCT No.: PCT/TR2022/050144

§ 371 Date: Oct. 2, 2023

(30) Foreign Application Priority Data
Apr. 2, 2021 (TR) 2021/006002

(51) Int. Cl. A61N 1/20; A61N 1/04; A61N 1/32

(52) U.S. Cl. CPC A61N 1/205; A61N 1/0408; A61N 1/325; A61N 1/326; A61N 1/328

(57) ABSTRACT

A method of creating an antioxidant, antiapoptotic, antiaging, anti-inflammatory, antibacterial, antitoxic, energy metabolism regulating effect and balancing blood pH by creating chemical and biological reactions in human and animal body is provided. The method includes a circuit configuration that is created by serial connection of the human and animal body with resistance function to an electric circuit on which there is direct current and serially connected frequency generator to create chemical and biological reactions in the human and animal body, connecting the positively charged (anode) wire from the lung and/or abdominal area and/or legs and/or arms and connecting the negatively charged (cathode) wire to create a 180° angle on the back and/or legs and/or arms on the exact opposite of the point of connection of the positively charged wire in the body.

### CROSS REFERENCE TO THE RELATED APPLICATIONS

This application is the national phase entry of International Application No. PCT/TR2022/050144, filed on Feb. 18, 2022, which is based upon and claims priority to Turkish Patent Application No. 2021/006002, filed on Apr. 2, 2021, the entire contents of which are incorporated herein by reference.

### TECHNICAL FIELD

This invention is related to creating chemical (electrolysis in body fluids) and biological (creating an antioxidant, antiapoptotic, antiaging, anti-inflammatory, antibacterial and antitoxic effect, regulating the energy metabolism and balancing blood pH) reactions without damaging tissues by applying low doses of direct electric current to the human and animal body.

### BACKGROUND

Free atoms which are present in the human and animal body and called free radicals continuously need a supply of electrons due to the lack of electrons in their outer orbits. For this purpose, they steal electrons from the electron flow resulting from the creation of chemical reactions or physical movements, cells and intracellular organelles in the body and can cause damage to cells, intracellular organelles, tissues and other chemical reactions in the body. Such damage might lead to many diseases including cancer, Alzheimer's and Parkinson's.

They further steal electrons that are to be used in chemical reactions in the body and prevent the emergence of chemical reactions and they steal electrons from chemical reactions that are for recovery purposes in the body especially in infection conditions and prevent recovery from disease by disrupting reactions and lead to the spread of the disease. Even though the body generates antioxidants against these oxidants, a part of free radicals' need for electrons is met by antioxidants which are taken from outside of the body and known as vitamins. On the other hand, various effects such as antiapoptotic, antiaging, anti-inflammatory, antitoxic and energy metabolism regulation are tried to be created by various chemicals, drugs, sports and various diets.

In prior art, there is not any method or system that is similar to the problem solving approach developed by this invention—creating an antioxidant, antiapoptotic, antiaging, anti-inflammatory, antibacterial and antitoxic effect, regulating the energy metabolism and balancing blood pH through low doses of direct electric current.

### SUMMARY

### Purpose of Invention

This invention is a circuit configuration that is created by serial connection of the human and animal body with resistance function to an electric circuit on which there is direct current and serially connected frequency generator to create chemical and biological reactions in the human and animal body, connecting the positively charged (anode) wire from the lung and/or abdominal area and/or legs and/or arms and connecting the negatively charged (cathode) wire to create a 180° angle on the back and/or legs and/or arms on the exact opposite of the point of connection of the positively charged wire in the body, and with the electrons generated in the body by the electric current going through the circuit, wherein it is intended with the electrons to;
support the immune system by creating an antioxidant effect by meeting the need for electrons of free radicals and/or cells and/or cellular organelles with an electric current,
provide the necessary electron supply for the chemical reactions in the body, stop cell deaths and proliferation of carcinogenic cells by providing electrons to cells and organelles which have lost electrons in their structure, create a ceasing effect on cell destruction/death (antiapoptotic) by completing the lack of electrons in cells which have lost electrons,
eliminate tissue and cell damages and create an antiaging effect as these antioxidants become more supportive in cell and tissue activities because free radicals which reach a steady state with the electrons provided from the circuit need fewer antioxidants generated in the body,
create an impact on adjustment of blood pH by creating hydroxide (OH⁻) and water (H₂O) by making a covalent bond with the help of the electrons carried to the body with the electric current with free hydrogen (H⁺) and free oxygen (O⁻) on occasions when blood pH balance is disrupted and acidosis occurs in humans and animals, with the energy and electrons provided from the circuit,
create an impact on adjustment of blood pH by creating water (H₂O) by making a covalent bond with the help of the electrons carried to the body with the electric current with free hydrogen (H⁺) and free hydroxide (OH⁻) on occasions when blood pH balance is disrupted and alkalosis occurs in humans and animals, with the energy and electrons provided from the circuit,
create an antibacterial effect which provides the death of bacteria with the chemical change created by the energy and electrons provided by the electric current going through the circuit,
create an antibacterial effect which provides disintegration in the cell membrane of bacteria and therefore, for the death of bacteria with the energy and electrons provided by the electric current going through the circuit,
create an anti-inflammatory effect by reducing proinflammatory cytokine releases such as tumor necrosis factor alpha (TNF-α) and interleukins by disabling bacteria in order to prevent their spread in the body, isolate and disable them.

Furthermore, with the energy created by the electric current going through the circuit, the invention intends to;
create an effect of increasing glucagon hormone release to convert glycogen into glucose and thus, use it as an energy source and regulate the energy metabolism depending on the increasing energy consumption in the body.

The invention is related to an electric circuit configured to make chemical and biological changes needed in the human and animal body.

The invention is an electric current configuration comprising of the serial connection of a) a direct current generator that converts alternative current to direct current, b) and/or a direct current generator that has a direct current accumulator/battery, c) an electrical frequency generator, d) a human/animal body that functions as variable resistance with the impact of the frequency generator, e) an on/off switch and f) the concerned components with a conductive wire.

Moreover, the invention has a structure that ensures the electric current coming from the direct current generator does not harm the body by changing the resistance of the human body that functions as resistance in the circuit to electric current through the frequency generator and thus, reaches the highest current with the lowest voltage.

The invention brings them to a steady state (antioxidant effect) by completing the missing electrons in the outer orbits of free atoms which are present in body fluids and called as free radicals that have a lack of electrons in their outer orbit with the electrons that enter the body through the electric current. Therefore, the invention prevents these atoms from damaging cells by stealing electrons from cells, disrupting chemical reactions by stealing electrons from body fluids, and also development of carcinogenic cells.

Furthermore, the invention provides free radicals to use less antioxidants that are generated in the body by meeting the electron need of free radicals and slows down aging of cells and tissues (antiaging) through the use of these antioxidants in strengthening of cells and tissues.

The invention, with the electrons carried to the body with the electric current, ensures;
The amount of antioxidants generated in the body increases by meeting the electron need of free radicals and the mitochondrial activity is supported with antioxidant support to cells and tissues,
Cell death is stopped (antiapoptotic effect) by changing apoptosis that occurs based on overexpression of proapoptotic proteins such as Bax in many degenerative diseases and the balance between proapoptotic (Bax) and antiapoptotic (Bcl-2) proteins in favor of antiapoptotic (Bcl-2).

The invention, with the electrons carried to the body with the electric current in humans and animals, ensures;
Cell membrane and cellular organelles which lost electrons receive electrons, and accordingly, the cell turns back to its healthy state, therefore, tissue damage caused by free radicals and inflammatory effects is eliminated,
Damage that can be caused in the tissue by hypoxia that occurs as a result of reduced blood flow in the tissue (ischemia) is alleviated with increased oxygen saturation depending on the electric current.

The invention provides generation of hydroxide (OH⁻) and water (H₂O) by the covalent bond of free hydrogen (H⁺) and free oxygen (O⁻) thanks to the electrons carried to the body with the electric current and thus, correcting blood pH value on occasions when blood PH balance is disrupted and acidosis occurs in humans and animals.

The invention provides generation of water (H₂O) by the covalent bond of free hydrogen (H⁺) and free hydroxide (OH⁻) thanks to the electrons carried to the body with the electric current and thus, correcting blood pH value on occasions when blood PH balance is disrupted and alkalosis occurs in humans and animals.

The invention, with the electric current supplied from the circuit, in humans and animals;
Increases the release of glucagon hormone to convert glycogen into glucose and thus, use it as an energy source, depending on the increased energy consumption in the body.

The invention, with the electric current supplied from the circuit, in humans and animals;
Provides dissociation of the bonds by detaching the electron that creates the covalent bond of hydrogen atoms from its orbit by increasing its energy depending on the electric current that is supplied externally in toxins which are present in body fluids and have carbon (C), hydrogen (H) and/or nitrogen (N), hydrogen (H) covalent bond in their structure, and bringing the new molecule to a steady state thanks to the new electrons supplied to the environment with the electric current to the outer orbits of nitrogen (N) and/or carbon (C) atoms from which hydrogen (H) is detached and thus, making it lose its toxic character (antitoxic effect).

The invention, with the energy created with the impact of the electric current in body fluids after the electric current supplied from the circuit in humans and animals, ensures;
Sodium chloride (NaCl), potassium chloride (KCl) and water (H₂O) decompose as sodium (Na), potassium (K), chlorine (Cl), hydroxide (OH⁻) and hydrogen (H),
Sodium hydroxide (NaOH), potassium hydroxide (KOH) and hypochlorous acid (HOCl) are created with the electrons supplied to the environment,
The membrane structure of bacteria degrades and dissociates by decomposing the membrane of bacteria present in body fluids by sodium hydroxide (NaOH) or potassium hydroxide (KOH),
Bacteria are disintegrated when the membrane of bacteria present in body fluids is decomposed by hypochlorous acid (HOCl) and thus its membrane structure is disrupted,
Leukocytes' ability to create extracellular traps increases due to Ca⁺² increase in the cell when human monocyte derived macrophages are exposed to HOCl,
Immunity is developed (antibacterial effect) when genetic materials (RNA and/or DNA) that transfer to the body fluid after bacteria are disintegrated are obtained by Memory T cells.

The invention creates an anti-inflammatory effect by reducing proinflammatory cytokine releases such as tumor necrosis factor alpha (TNF-α) and interleukins by disabling the pathogen to prevent the spread, isolate and disable bacteria in the body, created with the impact of the bacteria that are disabled due to chemical and biological effects created with the electric current supplied from the circuit in humans and animals.

The invention, with the energy effect on the body of the electric current supplied from the circuit in humans and animals and the electrons supplied to the body, as demonstrated in the chemical formula “glycogen (C₂₄H₄₂O₂₁)+Energy→Lactate (C₃H₆O₃)+Water (H₂O)” ensures;
Hydrogen bonds of glycogen separate and reduce to lactate level,
These hydrogens and hydroxides separated from glycogen bond with the electrons supplied to the environment and create H₂O,
Lactate (C₃H₆O₃) transfers to the mitochondria and turn it into biological energy.

### DETAILED DESCRIPTION OF THE EMBODIMENTS

With an aim to analyze the contributions of the invention, in the bacterial sepsis model experimentally created in rat, the impact of application of a low dose of direct electric current on a) inflammation response, b) blood bacteria count, pH, gas, viscosity, cell count and some biochemical parameters, c) oxidative stress, d) tissue damage was analyzed. These analyses are detailed below.

Preparation of bacteria suspension: Commercially supplied Staphylococcus aureus standard strain (ATCC-10390) was used in analyses. Passages were taken from the strains kept lyophilized at −20° C. before injections and incubated at suitable times. To ensure that no contamination or structural change occurred in the standard strains, identification was made in certain time intervals using VITEK 2 Compact 30 automatic micro identification system and VITEK MS mass spectrophotometry (bioMérieux, Lyon, France). As a result of identification, it was determined that the strains demonstrated a similarity of 99.9%. Moreover, in order to ensure the standard strain did not mutate and did not lose its pathogenicity, only the bacteria obtained from the initial passages were used at all times. An effort was made to ensure that the standard Staphylococcus aureus strains had reproduction in the logarithmic phase during the experiment. For this reason, the passage of strains was taken 18-24 hours before injection and incubated in a stove at 37° C. during this period of time. After incubation, the bacteria suspension in suitable density was prepared according to the McFarland turbidity system (1×10⁹ KOB KOB/ml) using DensiCHEK plus (bioMérieux, Lyon, France). sterilACILA® LAL ReagentWater (LRW, Opelstrassee 14, Mörfelden-Walldorf, Germany) was used for preparing the suspensions. These suspensions were drawn into 1 ml injectors on sterile conditions and made ready for application. In order to determine the reliability of our sepsis model at the end of the experiment, microbiological examinations were made using the culture method in blood samples.

Determining the Culturable Bacteria Count: Blood samples (100 μl) that were taken 1 and 6 hours after bacteria injection were left for 3 hours of pre-enrichment in 10 mL commercial TSB tubes (BD, BBL Trypticase Soy Broth, LOT: 01896243, Franklin Lakes, New Jersey, USA). At the end of 3 hours, 100 μl was taken and, using the spread plate method, implanted in 3 repetitions in Mannitol Salt Agar (Merck-Millipore, LOT: 146023, Darmstadt, Germany). Cultivated media were left for incubation for 72 hours at 37° C. Colony forming units were counted at the end of this period and reproduction results were recorded.

Device used for applying electric current: This recently developed low electric current application device (Dr Biolyse) is an electron accelerator that can be used to dissociate chemicals in body fluids. The operating principle of the device is separating molecules with the energy supplied by the electric current and creating new molecules with the electrons supplied to the environment. There are 3 types of compounds in body fluids that have close molecular bonds and thus, can easily be disintegrated and compounded due to fire. These are water (H₂O), sodium chloride (NaCl) and potassium chloride (KCl). For this reason, we thought before this experiment that these compounds can be disintegrated and new compounds can be created from the foregoing chemicals in body fluids with an energy and electron transfer caused by exposure to a low dose of direct electric current (electrolysis) without increasing the body temperature. To test this idea, we applied direct electric current at a low dose to increase the orbital speed of the electrons that are commonly used in covalent bonds in in vitro isotonic saline solution and thus, we increased the average rotating speed depending on the speed at which the electron detaches from the orbit to create resonance with the electric frequency. By doing so, high efficiency detachment was scored by applying low dose direct electric current at 300 Hz frequency to reach a speed of 300.000 km/second that is the approximate speed of the electron after detachment. In this process, H₂O broke down to hydrogen (H) and hydroxyl (OH) ions and NaCl broke down to sodium (Na) and chloride (Cl) ions. While the reaction was continuing, sodium hydroxide (NaOH), H and Cl were created in the environment. After NaOH generation reached a point, instant hypochlorous acid (HOCl, a weak acid) was generated. This indicated that the system started a rapid balancing in certain periods of time. The balance reaction was NaOH+HOCl→NaCl+H₂O+O.

In the analyses, two pairs of carbon electrodes were placed on the lungs and back, abdomen and back to measure the resistance to be generated by the body of the rat against the electric current. When the rat body resistance was measured by applying a standard direct electric current, it was between 135,000-150,000 Ohm. Applying such direct electric current continuously causes tissue damage after a while due to the load it creates on cells. On the other hand, when it was measured with a Dr Biolyse device which had a square wave frequency, the body resistance at 300 Hz frequency was between 2,700-3,300 Ohm. This 300 Hz frequency was selected based on our prior in vitro experiment as it offered the most efficient resonance on H₂O, NaCl and KCl and made molecular dissociation at the fastest level. With this frequency setting, the contact of the electric current with tissues was kept at the minimum and the current was transferred to body fluids. By doing so, the electric current reached the target fluid environment with a small resistance on body tissues.

Animals Used and Creating Sepsis: As a result of the power analysis (10 unit difference in average and 5 unit standard deviation at 80% power and 95% confidence level) it was determined that minimum 7 rats were necessary for each group. For this reason, 28 male Wistar albino rats that weighted 250 to 300 grams were used in this study. The rats were obtained from Bezmialem Foundation University Experimental Animals Center. The animals were kept at standard temperature (25±1° C.), humidity (50-60%) and lighting conditions (12 hour light/12 hour dark cycle) and fed ad libitum. The ethical approval was obtained from the Bezmialem Foundation University Experimental Animals Center Ethics Board. The rats were grouped into 4, namely healthy control (K), electrified healthy (E), sepsis (S) and electrified sepsis (SE). Before the experiment, to place the electrodes, the rats' abdomen and back areas were shaved with an electric shaver. To create sepsis, the animals in groups S and SE were injected Staphylococcus aureus (1×10⁹ CFU) at 1 ml LRW (i.p.). The animals in groups K and E were injected 1 ml LRW in place of Staphylococcus aureus. Sepsis formation was verified by the bacterial implantation of 100 [symbol] l blood taken from the jugular vein of the rats with isoflurane anesthesia 1 after bacteria or LRW injections.

To apply low dose electric current (300 Hz and 2.5 volt), positive electrodes were placed on the chest and abdomen of the rats and negative electrodes were placed on the back at the exact opposite of their chest and abdomen, therefore, the electric current went through the body for the electrolysis of the body fluids. The electrodes were fastened on the rats' back and abdomen with a plaster band completely wrapped around the body. The electric current was applied to the rats in groups E and SE for 40 minutes 1 and 6 hours after the bacterial injection. The electrodes were placed on the same areas for 40 minutes without applying any electric current in the rats in groups K and S. At the end of this period, the rates were anesthetized with ketamine-xylazine (100 mg/kg-10 mg/kg) and blood and tissue samples were collected to be studied later.

Histopathological Examination: The obtained tissues were fixed in 10% buffer formaldehyde for light microscope studies. The samples in buffered formaldehyde were processed in a tissue monitoring device (Leica TP 1020, Germany) under constant vacuum and embedded in paraffin. 3-5 micrometer thick paraffin sections were taken from paraffin blocks and then, stained with hematoxylin-eosin stain for histopathological study. Each section were independently evaluated by two histologists that were blind to the groups in 10×, 20× and 40× magnification. The images were taken by a Nikon digital camera (Eclipse 920248, USA).

Whole Blood Counts: Whole blood cell count was measured using a hematology analyzer (Abacusjuniorvet, Budapest, Hungary). Blood gas and pH were measured using a blood gas analyzer (ABL90FLEX, Bronshoj, Denmark). Whole blood viscosity and shear stress were measured using Wells-Brookfield cone plate viscometer (DV3TLVCJ0, USA) at 1,500 s⁻¹ shearing speed. All viscosity measurements were made at 37° C.

### Biochemical Analysis

1. Obtaining samples: Blood samples were centrifuged for 10 minutes at 3000×g to take serum supernatant. Tissue samples were homogenized using a homogenizer (Fast prep-24, MP Biomedical, USA) with PBS (Phosphate Buffer Saline, pH: 7.4). Serum and tissue samples were kept at −80° C. until experiments.
2. Analysis: In serum samples, cholesterol, aspartate amino transferase (AST), alanine amino transferase (ALT), lipase and total protein (TP) levels were measured with a semi automatic biochemical analysis device (IDEXX Vettest, IDEXX Laboratories, Inc. USA). For the kits, the measurement ranges were 20-161 U/L for ALT, 39-111 U/L for AST, 10-150 U/L for lipase, 20-92 mg/dL for cholesterol and 5.3-6.9 g/dL for TP.

### Analysis of Oxidative Stress Parameters

1. Measurement of Superoxide Dismutase (SOD), Malondialdehyde (MDA) and Reduced Glutathione Levels: In the study, in serum and lung and liver tissue homogenates, SOD (Elabscience Co., USA, Catno: E-EL-R1424), MDA (Mybiosource Co., USA, Catno: MBS738685) and glutathione (Elabscience Co., USA, Catno: E-BC-K030-M) levels were measured with commercially available ELISA based kits. Briefly, the standards and samples were pipetted to plates covered with monoclonal antibody, then left to micro plate incubation. All wells were added biotin and Streptavidin-HRP was added for bonding. After incubation, they were washed 4 times to remove those which were not bonded. After adding chromogenic solutions A and B, the stop solution was added to stop the reaction. The optical density was measured in plate reader at 450 nm wavelength (Thermo Scientific Microplate Reader). The identification range of the kits were between 0.16-10 ng/mL for SOD, 0-1000 ng/mL for MDA and 2-400 μmol/L for Reduced Glutathione.
2. Measurement of total antioxidant capacity (TAS) and total oxidant capacity (TOS) levels: In the study, TAS levels in serum samples and lung and liver tissue homogeny were measured with the spectrophotometry method using a commercial kit (RelAssay Diagnostics, Turkey). Briefly, the antioxidants in the samples reduced dark blue green color ABTS radical to a colorless reduced ABTS form. Absorbance change at 660 nm was related to the total antioxidant level of the sample. Total antioxidant activities were expressed in mmolTroloxEquiv/L of samples.

Furthermore, TOS levels in samples were measured again with the spectrophotometry method using a commercial kit (RelAssay Diagnostics, Turkey). Briefly, the oxidants in samples oxidized ferrous ion chelator complex to the ferric ion. While the oxidation reaction was prolonged with intensifier molecules which were in plenty amounts in the reaction environment, the ferric ion created a color complex with the chromogen in the acidic environment. This color density was related to the total amount of oxidant molecules present in the samples. The results were expressed in μm H₂0₂ Equiv/L.

Measurement of multiplex inflammation parameters: In the study, the cytokine parameters in serum samples were measured with the commercially available inflammation panel (Biorad, USA, BioplexRatcytokineplexassay, catno: 12005641). The panel had 30 parameters. The test installation was as recommended by the manufacturer. Briefly, the samples were mixed with magnetic beads connected to the antibody on a 96 well plate and shaken and incubated for the night at 4° C. The incubation steps in cold and at room temperature were realized in an orbital shaker at 500-600 rpm. The microplate was washed twice with the washing buffer. After 1 hour of incubation at room temperature with biotinylated identification antibody, streptavidin was added. The plates were washed as above and PBS was added with a lower threshold of 50 beads per sample. Reading was performed with Bio-Plex 200.

Sodium dodecyl sulphate-polyacrylamide gel electrophoresis (SDS-PAGE) and Western Blot Analysis: After the tissues were dissolved, they were cut into pieces of about 100 mg, and homogenized using a bead homogenizer for 10 minutes in 500 μl cold RIPA buffer (Santa Cruz Biotechnology, Dallas, Texas, USA) that included a protease inhibitor cocktail. The homogenate was centrifuged at 4° C. for 15 minutes at 700×g to remove residues and seeds and the supernatant was collected in 1.5 ml tubes and groups were pooled. The samples were kept at −80° C. until further experiments. The supernatants of tissues were denatured and separated using a 12% sodium dodecyl sulphate polyacrylamide gel. The proteins (20 μg) were transferred to a polyvinyl difluoride membrane (Bio-Rad, Hercules, CA, USA). The membrane was blocked with 5% skimmed milk powder (Bio-Rad, Hercules, CA, USA) in a tris (hydroxymethyl) aminomethane (Tris) buffer saline that included 0.1% Tween 20 (TBST). After that, it was immuno blotted with primary antibodies (IL-1p, TNF-a, Bax, Bcl-2, tubulin and β-actin (Cell Signaling, Danvers, Massachusetts, USA)) for the night at 4° C. After washing with TBST solution three times, the membranes were incubated for 1 hour at room temperature with horse radish peroxidase (HRP) and conjugo secondary antibodies (goat anti-rabbit immunoglobulin G (IgG) HRP and goat anti-rat IgG HRP (Cell Signaling, Danvers, Massachusetts, USA). Western blots were developed with peroxidase reaction with ECL reactives (Elabscience, Houston, Texas, USA) and displayed with Fusion FX7 system (Vilber Lourmat, France). Band density measurement was made using ImageJ software (National Health Institutes, Bethesda, MD, USA).

Statistical analysis: Shapiro-Wilk test was used to test if data had normal distribution. Normally distributed data were evaluated with one way ANOVA test and not normally distributed data were evaluated with Kruskal-Wallis test. Post-hoc comparisons among groups were made with Bonferroni and Dunns tests using GraphPadPrism software (GraphPadPrism Version 6 Software San Diego, CA). Group averages were calculated as ±standard deviation (SD) for all values. P<0.05 value was considered statistically significant.

### Results

Bacteria Count in Blood: One hour after LRW or Staphylococcus aureus injection, right before applying electric current, bacteria reproduction was not displayed in blood in groups K and E. However, there was intense bacteria reproduction in blood in groups S and SE which demonstrated bacteremia (bacteria reproduction in blood) in animals in these two groups. Applying electric current for 40 minutes at 1 and 6 hours after the bacteria injection significantly reduced the bacteria count in SE group.

Tissue Histology: When the histologic results of the groups were examined, there was inflammation in the myocardium of group S while there was no inflammation in the myocardium of group SE. All groups other than group S had normal histological structures. It was observed that the myocardial fiber bundle was loose and some myocardial fibers were necrotic in group S.

The histologic study of the lungs demonstrated that groups K and E did not have pulmonary histologic changes. Group S had inflammatory cell infiltration. Group SE had also inflammation but much less than that of group S. Additionally, congestion was higher in group SE than all other groups.

With respect to liver tissues, there was no inflammatory cell proliferation in any group. All groups other than group S had normal histological structures. On the other hand, sinusoids were slightly larger in all groups due to fixation. Group S tissues had fields of necrosis.

Changes in Blood Parameters: When compared to group K, in group S, pH, oxygen saturation (sO₂) and white blood cells (WBC) were significantly lower and pCO₂, red blood cells (RBC), hemoglobin (HB) value, hematocrit percentage (HCT %), blood viscosity and shear stress values were significantly higher. On the other hand, none of the changes that occurred in group S were observed in group SE. The groups did not have significant difference in terms of thrombocyte (PLT) count, monocyte percentage or pO₂. Moreover, compared to group K, in group E, lymphocyte percentage significantly increased and granulocyte percentage and RBC significantly decreased.

Biochemical Changes in Blood: Compared to group K, in group S, serum ALT and AST levels and lipase enzyme activity were significantly higher and TP level was significantly lower. However, none of the changes that occurred in group S were detected in group SE.

Oxidative Stress Results: Compared to group K, in group S, blood and tissue TOS and MDA levels were significantly higher and TAS and GSH levels and SOD enzyme activity were significantly lower. On the other hand, none of the changes that occurred in group S were detected in group SE.

Changes in Cytokine Levels in Blood: When serum inflammation indicators were analyzed, all interleukins (ILs) and macrophage inflammatory proteins (MIPs) other than IL-5, IL-13 and MIP-3α significantly increased. Moreover, granulocyte colony stimulating factor (G-CSF) and macrophage colony stimulating factor (M-CSF) were significantly lower, interferon gamma (IFN [symbol] ), monocyte chemo attractant protein 1 (MCP1) and chemokine ligand (CCL5 or RANTES) were significantly higher. Moreover, all these parameters in group S did not change in group SE other than IL-1α which was still higher in group SE compared to group K.

Western Blotting: Inflammatory cytokines in liver, heart and lung tissues of rats and changes in IL-1 [symbol] and TNF- [symbol] levels are demonstrated. In all tissue types analyzed, the expressions of these cytokines were upregulated in group S compared to group K. Electric current treatment significantly reduced that. Moreover, electric current treatment applied to healthy rats did not cause any change in the expression of these inflammatory cytokines in liver, heart and lung tissues.

Due to the relationship between increasing proinflammatory cytokine levels in sepsis process and apoptosis, the ratio of Bax (pro-apoptotic member of Bcl-2 family) to Bcl-2 (anti-apoptotic member of Bcl-2 family) was analyzed by using indicators for apoptosis rate. In liver and heart tissues, the Bax/Bcl-2 ratio pointed at an increase in apoptosis and was significantly higher in group S compared to group K. However, the Bax/Bcl-2 ratios of group S lung tissues did not change compared to group K. Electric current treatment significantly reduced apoptosis ratio in liver, heart and lung tissues in group SE compared to group S. Additionally, electric current treatment applied to healthy rats did not cause any change in apoptosis in the examined tissues compared to group K.

Analysis Result: Histologically, preservation of the tissue integrity and absence of necrosis demonstrated that applying an electric current at a low dose did not cause any damage to the tissues or organs of the rats. Despite the fact that inflammation was present in lung tissues of both groups of S and SE, inflammation was much lower in group SE compared to group S. This showed us that the rats were successfully infected experimentally, but low dose electric current significantly reduced the spread of infection in the lung tissue.

In our study, compared to group S, in group K, pH, SO₂ and leukocyte (WBC) count were lower and PCO₂, erythrocyte (RBC) count, HB value, HCT %, blood viscosity and shear tension levels were higher. Similarly to our findings, it was suggested that blood pH drops in severe sepsis. The changing pH and blood gas levels in group S might result from the relationship of sepsis with multiple organ failure that can lead to respiratory and circulation disorders and acute kidney failure.

Blood viscosity is a measure of blood fluidity and can be defined as the rate between shear tension and shear speed. It can increase in acute inflammatory diseases due to increasing acute phase proteins. HCT % can also change blood viscosity. In our study, the increased blood viscosity in septic rats compared to controls might be a result of increased RBC, HB and HCT %. Respiratory (hypoventilation) and circulation (hypoperfusion) failures in sepsis can lead to tissue hypoxia. For this reason, hypoxia induced erythropoietin generation can be the reason for increased RBC, HB and HCT % in septic rats compared to controls. WBC significantly reduced in group S compared to group K. The reduced WBC count can be related to immunological paralysis. This situation is an important character of severe sepsis and leads to increased mortality. On the other hand, none of the changes that occurred in group S were observed in group SE. Moreover, the low electric current increased the lymphocyte percentage and lowered the granulocyte percentage and RBC in group E compared to group K. Our experiment is the first experiment to show such impact of low electric current on lymphocyte and monocyte percentage.

Serum ALT and AST levels and lipase enzyme activity were higher while TP was lower in group S compared to group K. Increased lipase activity is an indicator of pancreatitis or septic shock and increasing ALT and AST levels and decreasing TP level is an indicator of liver damage. Applying low electric current prevented all these changes in group SE.

Previous studies demonstrated that lung and kidney injuries occurred as a result of sepsis due to excessive reproduction of reactive oxygen species (ROS) such as MDA, neutrophil accumulation and increased pro-inflammatory cytokine generation in tissues. ROS levels are neutralized and kept in balance by the body's antioxidant defense systems such as GSH and SOD. If this balance is disrupted in favor of ROS, destructive reactions occur in molecules such as proteins, lipids and nucleic acids. This situation called “oxidative stress” eventually causes tissue damage. Blood and tissue levels of TOS and MDA were higher and TAS and GSH levels and SOD enzyme activity were lower in group S compared to group K. This situation demonstrated that the oxidant/antioxidant balance disrupted in favor of the oxidant and therefore, presence of oxidative stress. However, applying low electric current prevented formation of oxidative stress that displayed antioxidant effect in group SE.

In sepsis, many ILs increase. In parallel to previous studies, all ILs examined in our study other than IL-5 and IL-13 increased in group S compared to group K. It was reported that low IL-5 level is related to lung death and tissue damage and thus, IL-5 treatment can reduce sepsis related mortality rate. A study demonstrated that IL-13 protected from the mortality of sepsis by modulating the inflammatory responses by suppression.

Our study also demonstrated that all MIPs examined other than MIP-360 increased in group S compared to group K. In the literature, there is limited information available about MIP-3α level and the results of other MIP families in sepsis are controversial. Use of G-CSF is recommended in sepsis patients to increase myeloid cell functions. Interestingly, while GM-CSF value was expected to drop in group S, it was higher than controls in our study. This result might be related to an early or late phase of sepsis.

In our study, GRO/KC, MCP1 and RANTES were higher in group S compared to group K. To our knowledge, there is not any study on GRO level in sepsis, but there are many studies on cancers. It was reported that GRO levels increased in different types of cancer. MCP1 is a strong chemo attractant present in various inflammatory diseases and it is a regulatory intermediary. RANTES also has a similar effect. On the other hand, applying electric current showed an anti-inflammatory effect by preventing the development of inflammation indicators other than IL-1 [symbol] in group SE. IL-1α is generated by active macrophages and neutrophils, epithelial cells and endothelial cells. It plays an important role in regulating immune responses by bonding to interleukin-1 receptor. Higher IL-1 [symbol] in group SE compared to group K can depend on bacteremia, even a little, detected in group SE. In our study, low electric current was applied for forty minutes in two sessions. Probably, bacteremia can be eliminated by increasing the number of sessions. This also explains the presence of inflammation, even a little, in the lungs in group SE. The relationship between sepsis and increased pro-inflammatory cytokines such as TNF-α and IL-1β is known as a mechanism that is enabled to eliminate invader pathogens.

On the other hand, these immune system regulators play a role in sepsis induced pathophysiology by encouraging inflammation that causes excessive tissue damage. In this study, the upregulation of TNF-α and IL-1β, the most comprehensively examined cytokines in sepsis pathophysiology was recorded in liver, heart and lung tissues of rats. On the other hand, low electric current reduced these cytokine levels to a level close to control values by recovering the tissue damage caused by sepsis. In previous studies, the increase in proinflammatory cytokines in lung tissues due to sepsis was reduced by electrical stimulation. Furthermore, researchers found that electrical stimulation of the vagus nerve during sepsis had a weakening effect on the TNF-α level.

Cell death increases due to activation of the mitochondrial pathway of apoptosis during sepsis. Our recently developed low dose direct electric current application method also enables an anti-inflammatory mechanism to eliminate sepsis induced tissue damage.

In this study, we found an increase in the apoptotic speed of the liver and heart tissue of the animals with sepsis, which is consistent with recent studies. The induction in the pathophysiological process of sepsis can result from both an apoptosis induced decrease in immune cells and the immunosuppressive effect of apoptotic cells. Previous studies demonstrated that treatments that inhibit apoptosis can be an effective strategy against sepsis induced tissue damage. Moreover, low dose electric current reduces the apoptosis rate by increasing the chance of cell survival after sepsis. Furthermore, it was found that electrical stimulation had a positive impact on kaspaz-3 and Bax pulmonary expressions as a compensatory mechanism against apoptosis.

In summary, low dose electric current application has antibacterial, antioxidant, anti-inflammatory and antiapoptotic effects due to the electrolysis it creates in body fluids without causing any damage to body tissues.

### APPLICATION OF INVENTION TO INDUSTRY

The invention is a method used for creating chemical and biological effects in humans and animals and can easily be applied to industry.

What is claimed is:

1. A method of creating an antioxidant, antiapoptotic, antiaging, anti-inflammatory, antibacterial, antitoxic, and energy metabolism regulating effect and balancing a blood pH by creating chemical and biological reactions in human and animal body without damaging tissues by applying low doses of a direct electric current to the human and animal body, comprising: serial connecting a direct current generator, an electrical frequency generator, a human/animal body, an on/off switch, and concerned components with a conductive wire, wherein the direct current generator is allowed to convert an alternative current to a direct current and/or the direct current generator has a direct current accumulator/battery, and the human/animal body functions as a resistance,
a configuration, wherein the configuration is allowed to change the resistance of the human/animal body in a circuit to an electric current through the electrical frequency generator to ensure the electric current coming from the direct current generator does not harm the human/animal body and thus to reach a highest current with a lowest voltage, and
a circuit configuration, wherein the circuit configuration is created by connecting a positively charged (anode) wire from a lung and/or an abdominal area and/or legs and/or arms and connecting a negatively charged (cathode) wire on a back and/or on an opposite of a point of a connection of the positively charged wire in the human/animal body to create a 180° angle.

2. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 1, further comprising: using an energy created with an impact of the electric current in body fluids,
for a decomposition of sodium chloride (NaCl), potassium chloride (KCl), and water (H₂O) as sodium (Na), potassium (K), chlorine (Cl), hydroxide (OH), and hydrogen (H),
a creation of sodium hydroxide (NaOH), potassium hydroxide (KOH), and hypochlorous acid (HOCl) with electrons supplied to an environment,
a degradation and a dissociation of a membrane structure of bacteria by decomposing a membrane of the bacteria present in the body fluids by the sodium hydroxide (NaOH) or the potassium hydroxide (KOH),
a disintegration of the bacteria by a decomposition of the membrane of the bacteria present in the body fluids by the hypochlorous acid (HOCl) and thus disrupting the membrane structure of the bacteria,
ensuring an increase of Ca⁺² in a cell through an exposure of human monocyte derived macrophages to the HOCl, and thus increasing a leukocytes' ability to create extracellular traps, and
developing an immunity (antibacterial effect) when genetic materials (RNA and/or DNA) are obtained by Memory T cells, wherein the genetic materials (RNA and/or DNA) transfer to the body fluids after the bacteria are disintegrated.

3. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 1, further comprising: an ability to
create the antioxidant effect by meeting missing electron needs of free atoms to prevent the free radicals from stealing the electrons within the human/animal body, wherein the free atoms are present in the body fluids and called as free radicals, and the free radicals have a lack of the electrons in an outer orbit with the electrons passing through the electric current.

4. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 1, further comprising:
allowing an increase in an amount of antioxidants generated in the human/animal body by meeting an electron need of free radicals by the electrons carried to the human/animal body with the electrical current, wherein a mitochondrial activity and the antioxidant support to cells and tissues.

5. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 1, further comprising:
allowing a stop of a cell death (antiapoptotic effect) by changing apoptosis and a balance between proapoptotic (Bax) and antiapoptotic (Bcl-2) proteins in favor of the antiapoptotic (Bcl-2) by the electrons carried to the human/animal body with the electric current, wherein the apoptosis occurs based on an overexpression of proapoptotic proteins such as Bax in many degenerative diseases.

6. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 1, further comprising:
stopping impacts of aging (antiaging effect) and early cell death (antiapoptotic) by supplying the electrons to cell membrane and cellular organelles which lost electron, and accordingly, by providing a cell to turn back to a normal state by the electrons carried to the human/animal body with the electric current.

7. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 1, further comprising:
eliminating tissue damages by supplying the electrons to cell membrane and cellular organelles which lost electron, and accordingly, by providing a cell to turn back to a healthy state by the electrons carried to the human/animal body with the electric current.

8. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 1, further comprising:
allowing an alleviate of a damage with increased oxygen saturation depending on the electric current, wherein the damage is allowed to be caused in the tissues by a hypoxia, and the hypoxia occurs as a result of reduced blood flow in the tissues (ischemia).

9. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 1, further comprising:
generating hydroxide (OH⁻) and water (H₂O) by a covalent bond of free hydrogen (H⁺) and free oxygen (O⁻) thanks to the electrons carried to the human/animal body with the electric current and thus, correcting a blood pH value on occasions when a blood pH balance is disrupted and an acidosis occurs.

10. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 1, further comprising:
generating water (H₂O) by a covalent bond of free hydrogen (H⁺) and free hydroxide (OH⁻) thanks to the electrons carried to the human/animal body with the electric current and thus, correcting a blood pH value on occasions when a blood pH balance is disrupted and an alkalosis occurs.

11. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 1, further comprising:
increasing a release of glucagon hormone to convert glycogen into glucose with the electric current in the circuit and thus, using it as an energy source, depending on an increased energy consumption in the human/animal body.

12. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 1, further comprising:
dissociating bonds by detaching the electrons that creates the covalent bond of hydrogen atoms from its orbit by increasing its energy depending on the electric current supplied from the circuit in toxins, wherein the toxins are present in the body fluids and have carbon (C), hydrogen (H) and/or nitrogen (N), hydrogen (H) covalent bond in their structure.

13. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 1, further comprising:
bringing a molecule to a steady state by new electrons supplied to the environment with the electric current to outer orbits of nitrogen (N) and/or carbon (C) atoms, wherein hydrogen (H) is detached in toxins to make the toxins lose a toxic character (antitoxic effect), and the toxins are present in the body fluids and have carbon (C), hydrogen (H) and/or nitrogen (N), hydrogen (H) covalent bond in their structure.

14. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 1, further comprising:
providing an anti-inflammatory effect by reducing proinflammatory cytokine releases such as tumor necrosis factor alpha (TNF-α) and interleukins by disabling bacteria to prevent from spreading, isolating and disabling the bacteria in the human/animal body.

15. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 1, further comprising:
dissociating hydrogen bonds of glycogen and reducing bacteria to a lactate level,
creating H₂O by enabling hydrogen and hydroxides separated from glycogen bond with the electrons supplied to the environment,
transferring lactate (C₃H₆O₃) to mitochondria and turning the lactate (C₃H₆O₃) into a biological energy.

16. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 2, further comprising: an ability to
create the antioxidant effect by meeting missing electron needs of free atoms to prevent the free radicals from stealing the electrons within the human/animal body, wherein the free atoms are present in the body fluids and called as free radicals, and the free radicals have a lack of the electrons in an outer orbit with the electrons passing through the electric current.

17. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 2, further comprising:
allowing an increase in an amount of antioxidants generated in the human/animal body by meeting an electron need of free radicals by the electrons carried to the human/animal body with the electrical current, wherein a mitochondrial activity and the antioxidant support to cells and tissues.

18. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 2, further comprising:
allowing a stop of a cell death (antiapoptotic effect) by changing apoptosis and a balance between proapoptotic (Bax) and antiapoptotic (Bcl-2) proteins in favor of the antiapoptotic (Bcl-2) by the electrons carried to the human/animal body with the electric current, wherein the apoptosis occurs based on an overexpression of proapoptotic proteins such as Bax in many degenerative diseases.

19. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 2, further comprising:
stopping impacts of aging (antiaging effect) and early cell death (antiapoptotic) by supplying the electrons to cell membrane and cellular organelles which lost electron, and accordingly, by providing a cell to turn back to a normal state by the electrons carried to the human/animal body with the electric current.

20. The method of creating the antioxidant, antiapoptotic, antiaging, anti-inflammatory, antibacterial, antitoxic, and energy metabolism regulating effect and balancing the blood pH by creating the chemical and biological reactions in the human and animal body according to claim 2, further comprising:
eliminating tissue damages by supplying the electrons to cell membrane and cellular organelles which lost electron, and accordingly, by providing a cell to turn back to a healthy state by the electrons carried to the human/animal body with the electric current.
